(19) United States
(12) Patent Application Publication
(10) Pub. No.: US 2013/0296715 A1
(43) Pub. Date: Nov. 7, 2013

(54) INSTRUMENT AND METHOD FOR HIGH-SPEED PERFUSION IMAGING

(71) Applicant: ECOLE POLYTECHNIQUE FEDERALE DE LAUSANNE (EPFL), Lausanne (CH)

(72) Inventors: Theo LASSER, Denges (CH); Marc Andre, Bern (CH); Michael Friedrich, Bern (CH)

(21) Appl. No.: 13/935,947

(22) Filed: Jul. 5, 2013

Related U.S. Application Data
(63) Continuation-in-part of application No. 11/912,224, filed on Oct. 22, 2007, now Pat. No. 8,480,579.

(30) Foreign Application Priority Data
Apr. 20, 2005 (IB) 2005/051289

(51) Int. Cl. A61B 5/026

(52) U.S. Cl. CPC A61B 5/0261
USPC 600/479

(57) ABSTRACT

A high-speed laser perfusion imaging instrument including a laser source, a detector, a signal-processing unit, data memory, and a screen to display results. A section of a sample surface is illuminated with laser light; reemitted light from the irradiated surface is collected by focusing optics on a 2D array of integrating photodetectors having elements that can be accessed individually or in a pre-defined selection of pixels at high speed. This 2D array measures intensity variations at each individual pixel. Average amplitude and mean frequency of the measured signal contain information about concentration and speed of moving blood cells. For real-time imaging, exposure time is used as a parameter to measure relative perfusion changes. These data are stored and processed with the signal-processing unit to deliver 2D flow maps of the illuminated sample section, and allow a simple overlay between a conventional image and processed flow maps.

### CROSS-REFERENCE TO RELATED APPLICATIONS

This application is a continuation-in-part application of, and claims the benefit of priority under 35 U.S.C. §120 from, U.S. application Ser. No. 11/912,224, filed Oct. 22, 2007, herein incorporated by reference, which is a National Stage Application of International Application No. PCT/IB06/00940, filed Apr. 20, 2006. This application is based upon and claims the benefit of priority from prior International Application PCT/IB2005/051289, filed Apr. 20, 2005.

### FIELD OF THE INVENTION

The invention relates to imaging systems and more particularly to a perfusion and blood flow imaging system mainly applied for medical diagnosis.

### BACKGROUND OF THE RELATED ART

Laser Doppler Imaging (LDI) is a non-contact imaging modality based on the coherence properties of light. This imaging modality mainly developed thanks to new detector technology, software and the availability of appropriate laser sources. The performance improved steadily over the last two decades from the initial proposals based on a scanning instrument towards a state of the art instrument for medicine mainly due to a parallel imaging instrument based on CMOS array detectors.

LDI is a coherent imaging technique that allows imaging of moving particles especially cells in blood flow with a good discrimination between perfusion, flow velocities and the concentration of moving particles i.e. mainly the key flow parameters of erythrocytes.

In conventional scanning LDI the back reflected light from a biological sample or the skin or the organ is detected with a single point detector. This light contains the coherent superposition of a back reflected component from non-moving parts and a back reflected light component from moving particles which causes detectable light fluctuations and allows the extraction of maps of flow velocities, concentration of flow particles or the so-called perfusion as the product of flow velocity times flow particle concentration.

In parallel LDI, the signal results from the interference or coherent superposition between a coherent back-scattered light field originating from the coherently illuminated sample of non-moving parts and the coherent back-scattered light field from moving particles contained in the illuminated volume. A 2D array of random-pixel-access integrating photo detectors (e.g. integrating CMOS image sensor) is used to measure the intensity variations at each individual pixel. The average amplitude and the mean frequency of the measured signal contain information about concentration and speed of moving blood cells. Finally maps of flow velocities, concentration of flow particles or the so-called perfusion as the product of flow velocity times flow particle concentration can be displayed as an image.

Anomalous changes in peripheral blood flow are known to be an indicator of various health disorders in the human organism. Laser Doppler Perfusion Imaging (LDPI) is an imaging technique successfully used for visualization of two-dimensional (2D) micro-vascular flow-maps in a number of clinical settings including investigations of e.g. peripheral vascular diseases, skin irritants, diabetes, burns and organ transplants. This method is non-invasive because it involves no physical contact; the risk of infection and additional discomfort is completely avoided.

The technical principle is based on the Doppler effect wherein the light scattered by moving particles, e.g. blood cells, leads to a slight frequency shift, which can be measured by a heterodyne detector. A 2D flow map is obtained by means of sequential measurements from a plurality of predetermined points. In classical LDPI systems this is achieved by scanning the area of interest with a narrow collimated or focused laser beam. However this scanning approach is time-consuming and suffers from artifacts caused by the mechanical steering of the probing laser beam. In current commercial available LDPI systems these artifacts are circumvented on an expense of imaging time.

For those skilled in the art an alternative full-field flow imaging techniques using speckle contrast analysis is also known. For real-time full-field imaging, the exposure time is used as a parameter to measure relative perfusion changes by means of laser speckle imaging technique. The advantage of this approach is a fast image acquisition, which is achieved at an expense of spatial resolution. However, the technique can be hardly exploited for flow measurements, where either concentration or speed of moving particles is not known in advance. Both said parameters influence the system response in the same manner, and, generally, the cause of the contrast decay is not obvious. Also the system response is not linear to the velocity since a finite camera integration time influences the measurement.

In order to decrease the imaging time for parallel LDI, a parallel detection scheme has been employed increasing the imaging speed by a factor proportional to the number of channels working in parallel. A 2D matrix of photo-detectors is a suitable detection device for that purpose.

Recently Serov et al. [A. Serov, W. Steenbergen, F. F. M. de Mul, “Laser Doppler perfusion imaging with a complimentary metal oxide semiconductor image sensor”, Opt. Lett. 25, 300-302 (2002)] suggested a new approach on parallel laser Doppler imaging: a non-integrating true-random-addressing CMOS image sensor was used to detect Doppler signal from a plurality of points on the sample illuminated with a divergent laser beam. Here the mechanical scanning is substituted by the photoelectrical scan resulting in a faster imaging speed.

The use of non-integrating 2D array of photo-detectors for the purpose of laser Doppler has been disclosed in three publications.

A first publication U.S. Pat. No. 6,263,227: “Apparatus for imaging microvascular blood flow”. The concept of using a 1D or 2D matrix of conventional photo detectors is described. The imager can work in two modes—scanning or static. In the scanning mode a laser line is projected on the area of interest. The signals from the illuminated areas are detected by 1D matrix of photo detectors. By scanning the illumination laser light over the area of interest, a 2D perfusion map is obtained. In the static mode the whole area of interest is illuminated by an expanded laser beam or by light exiting an optical fiber. The Doppler signal is measured by 2D matrix of photo detectors. Each photo detector has its own electronics for signal processing. A CCD camera is used to observe the object of interest. The perfusion maps are superimposed on the photographic image obtained with the CCD.

A second publication WO03063677: “Laser Doppler perfusion imaging with a plurality of beams” and a third publication GB2413022: “Laser Doppler perfusion imaging using a two-dimensional random access high pixel readout rate image sensor”. Here, a structured illumination is used for illuminating a plurality of points or an area of interest. The Doppler signal from the illuminated areas is detected with 2D matrix of non-integrating (direct-access) photo detectors. For the detection, the use of random-access-fast-pixels-readout CMOS image sensor is claimed. A single CMOS image sensor is used for detecting the Doppler signal and to obtain a photographic image of the object of interest.

All previously mentioned publications describe arrays of non-integrating detectors that measure instantaneous changes of the photocurrent through the detector. Besides the fact that both publications disclose imaging systems based on integrating detectors, both documents use a true laser Doppler technique to measure the flow.

Laser speckle imaging (LSI) is an alternative technique to access blood flow in tissue. This technique has never been patented but was described in scientific publications; for a review see [J. D. Briers, “Laser Doppler, speckle and related techniques for blood perfusion mapping and imaging”, Physiol. Meas. 22, R35-R66 (2001)]. This technique is based on the image speckle contrast analysis. Various modifications of this technique were reported but those modifications are mainly focused on the signal processing part rather the measurement principal, which is virtually the same for all variants. The LSI system obtains flow-related information by measuring the contrast of the image speckles formed by the detected laser light. If the sample consists or contains moving particles, e.g. blood cells, the speckle pattern fluctuates. The measured contrast is related to the flow parameters (such as speed and concentration of moving particles) of the investigated object. The contrast value is estimated for a certain integration time (exposure time) of the sensor. The faster the speckle pattern fluctuations the lower the contrast value measured at a given exposure time. The control unit defines the exposure time of the image sensor to determine the range of the measured flow-related data related to the image contrast in Laser Speckle Imaging mode. Here, the integration time defines the range of measured speeds. The use of integrating image detectors is mandatory. Until now only the use of CCD type image sensors were reported for the technique.

The LSI is true real-time imaging technique, however as explained above the LSI signal approach cannot discriminate between concentration of flowing particles and their speed. The laser Doppler imaging provides more information as that the LSI method since with laser Doppler the concentration and speed signals can be measured independently. In LSI those signals are always intricately mixed i.e. it is impossible to deduce from speckle contrast changes in concentration or in speed of moving particles Generally, the LSI approach alone is more likely to be a qualitative indicator of blood flow but not a measuring instrument to accurately investigate physiological phenomena. However as claimed in this invention both concepts have the potential to be used in combination, which may lead to an even better overall performance for a perfusion imaging.

Summarizing the above considerations, we conclude:
a) LDI discriminates between the flow parameters (speed and concentration of moving particles). However LDI is perceived as a slower imaging modality in comparison to LSI.
b) LSI is a fast imaging technique, however the results obtained with this technique have not the information content as LDI i.e. does not allow acquiring particles speed and concentration independently.

### SUMMARY OF THE INVENTION

In this invention we describe an instrument that takes into account the advantages of both techniques for accurate and objective monitoring and the real-time imaging of microcirculation in tissue.

The aim of this invention is obtained thanks to a Laser Perfusion Imaging system comprising
a. at least one coherent light source,
b. a light collecting optic,
c. at least one 2D array of integrating photo detectors for receiving the collected light,
d. a control unit,
e. a signal processor unit and
f. a display unit to display the results,
   the coherent light source is arranged for illuminating a selected area of interest on a sample for determining flow-related data of said sample, said collected light photo detector being a two dimensional array of randomly addressed integrating photo detectors and is arranged for detecting a laser Doppler signal and/or image speckle signal from said selected area of interest of said sample.

### BRIEF DESCRIPTION OF THE DRAWINGS

The description will be better understood thanks to the attached drawings in which:

FIG. 1 describes a block diagram of the laser Doppler imaging system modules.

FIG. 2a shows the moment ratio M₁/M₀ (velocity) imager response on the change of the measured signal frequency.

FIG. 2b shows the (concentration, M₀) imager response on the change of the measured signal frequency.

FIG. 2c shows the (concentration) imager response on the change of the measured signal amplitude.

FIG. 2d shows a signal to noise ration of the system for measurements on finger and forearm skin. The standard deviations for each measured values are also shown in the graph.

FIG. 3 shows 256×256 pixels flow-related maps obtained with the new imager on finger skin

FIG. 3a shows the perfusion map

FIG. 3b shows the blood concentration map

FIG. 3c shows the flow speed map,

FIG. 3d shows the image of the object,

FIG. 4 shows time-sequence of images of an artery occlusion experiment,

FIG. 5 shows perfusion images obtained with the high-speed laser Doppler imager,

FIG. 6 shows an example of a coherent light source,

FIG. 7 shows a device for uniform diffuse illumination,

FIG. 8 shows a combination of LDI/LSI with a single image sensor,

FIGS. 9A-9B show implementations with two independent sensors for LDI/LSI,

FIG. 10 shows the x-axis LSI arbitrary perfusion units (apu) and the y-axis show LDI arbitrary perfusion units (apu).

### DETAILED DESCRIPTION OF THE INVENTION

An object of this invention is to propose an instrument for high-speed high-resolution imaging of microcirculation in tissues and to overcome the disadvantages of the prior described instruments or concepts.

A further object of this invention is a high-speed laser Doppler perfusion imaging system which allows digital photography, Doppler signal measurements and image speckle contrast analysis, all performed by a single detector.

An object of this invention is to acquire the signal from a plurality of illuminated spots by individual pixels, to integrate the induced photocurrent in a programmable, adapted way for increasing the signal-to-noise ratio and to process these signals for displaying finally 2D flow-related maps (perfusion, concentration, speed) with a high frame rate.

An object of this invention is to illuminate the sample i.e. biological tissue via a fiberized system in a very homogeneous way, by a fiber, GRIN-lens combination.

A further object of this invention is the use of a 2D matrix of integrating photo detectors that can be addressed randomly (pixel per pixel or in a ROI) with a high access rate. The integrating detectors organized in a 2D randomly addressed array allow a
i) Recording of the interferometric intensity fluctuations induced by detected dynamically scattered light individually for each detector element,
ii) Measuring of the contrast (blur) of the image speckles formed by the light reemitted from the object as a function of the integration time,
iii) Obtaining a digital photographic image of the object. This image is used for determining the anatomical boundaries associated with the blood flow-maps.

Another object of this invention is the description of a uniform homogeneous illumination of a section of an object of interest using a coherent light source such as an extended laser beam with a uniform intensity profile (see also the drawings FIGS. 6 & 7). The illuminating laser beam can scan the sample in a step-wise manner by a step-scanning system to increase the size of the measured area. The part corresponding to the illuminated region of the image received by the sensor is processed by the system. The backscattered light is collected with a light collecting optic on a 2D array of integrating detectors. Two approaches are used to analyze the signal:
i) the laser speckle approach allows performing full-field flow imaging in real-time;
ii) the laser Doppler technique is applied here to increase the obtained results.

Combination of these two techniques allows to decrease the total imaging time and to increase the accuracy of the measurement. Important, that both imaging techniques are performed with a single image sensor.

A further object of this invention is to use the integration time as an additional degree of freedom to measure flow parameters. The use of integrating detectors allows the increase of the photon collection efficiency, which results in an increased SNR (signal/noise ratio) of the measurements. That is of particular importance for the parallel detection concept (full-field detection). Also, the integrating detector allows the flexibility in selecting the integration time to always match the required signal bandwidth to the noise bandwidth reducing in this way the high-frequency noise contributions, therefore effectively increasing the SNR of the measurement.

A further object of this invention is the full-field illumination, where an area of interest is illuminated with an expanded laser beam. The illuminated surface is imaged on the matrix of integrating photo detectors via a light collecting optic with a certain (de-) magnification factor. A sequence of images is acquired during a certain data acquisition time; thus the history of the intensity variations is recorded into the memory for each pixel of the image in a digital format. The frequency content of this signal per pixel is analyzed with FFT algorithm. The total power of the intensity oscillations is proportional to the concentration of moving particles and to the integration time. Therefore the integration time is used as an additional parameter to estimate the speed. The frequency distribution of the intensity oscillations contains information about the speed distribution of moving particles.

A further object of this invention is the signal processing, which comprises the calculations of the flow-related signal (perfusion, concentration, speed) for each pixel of the image according to a predefined algorithm. The flow-related parameters are calculated from both the power spectra of the intensity fluctuations and the image speckle pattern contrast decay. Then, the flow-related maps are displayed on the monitor in real-time.

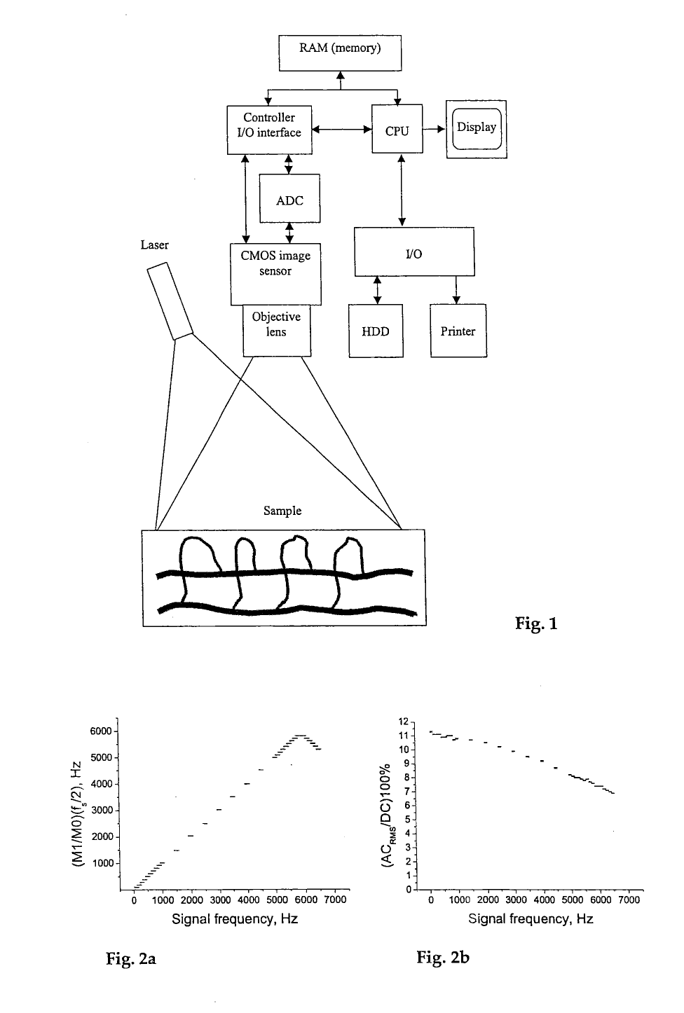

In FIG. 1, it is shown a block diagram of the laser Doppler imaging system modules. It comprises a laser source for illuminating the sample; the backscattered light is collected by an optic and detected by the CMOS image sensor. This signal is converted to a digital signal by the ADC converter and stored in the RAM memory. The control unit also called the Controller I/O Interface, ensures the necessary synchronization and settings of the CMOS image sensor and the link to the RAM memory as well as the signal processor unit or CPU unit. This CPU unit is also involved in the calculation and processing of the digital signal as well as the display onto a display unit and the data storage of the processed data across the I/O unit onto a hard disk HDD or printer.

In the memory (RAM) of the laser perfusion imaging system, in order to process the two type of detections, i.e. Laser Doppler and Laser speckle, two set of parameters are available. Other set of parameters can be available for standard imaging process, e.g. when acquiring the boundaries of the object of interest. The control unit CPU loads the selected parameters set and apply these parameters to the laser perfusion imaging system, i.e. to the light source, the collecting optics and the controller I/O interface. The CPU program related to the current processing is also loaded in the memory of said processing unit.

The signal sampling frequency is inversely proportional to the acquisition time of one sub-frame. The sub-frame sampling rate of the sensor depends on its size and the pixel clock frequency. In our case it was fixed at 40 MHz for the optimum performance speed/quality; higher pixel rates increase the noise level. The size of the sampled sub-frame finally defines the sampling frequency of the imager. E.g. for the sensor we used: for 256×4 pixels sub-frame the sampling frequency is 30 kHz, 256×6 pixels—20 kHz, 256×8 pixels—14 kHz, etc.

To obtain one flow map over a Region Of Interest (ROI), which is in our case 256×256 pixels, the ROI must be subdivided in smaller areas (e.g. in 32 sub-frames of 256×8 size) and scanned electronically. From 32 to 512 samples are obtained for each sub-frame, thus the intensity fluctuations history is recorded for each pixel of this predefined ROI.

The signal processing comprises the calculation of the zero-moment (M₀) and the first-moment (M₁) of the spectral power density S(ν) of the intensity fluctuations I(t) for each pixel. The zero-moment is related to the average concentration, <C>, of moving particles in the sampling volume. The first moment (flux or perfusion) is proportional to the root-mean-square speed of moving particles, Vᵣₘₛ, times their average concentration:

$$Concentration = 〈 C 〉 ∝ {M}_{0} = {\int }_{0}^{∞} S ( v ) d v$$

$$Perfusion = 〈 C 〉 {V}_{rm s} ∝ {M}_{1} = {\int }_{0}^{∞} vS ( v ) d v$$

$$S ( v ) = {\vert {\int }_{0}^{∞} I ( t ) exp ( - i 2 π v t ) d t \vert}^{2} .$$

Here ν is a frequency of the intensity fluctuations induced by the Doppler shifted photons. We calculated the power spectrum using FFT algorithm applied to recorded signal variations at each sampled pixel of ROI. The noise subtraction is performed from the calculated spectra by setting a threshold level on the amplitude of the spectral components. This filtering is applied to reduce the white noise (e.g. thermal and read-out noises) contribution to the signal. Thereafter the perfusion, concentration and speed maps are calculated and displayed on computer monitor.

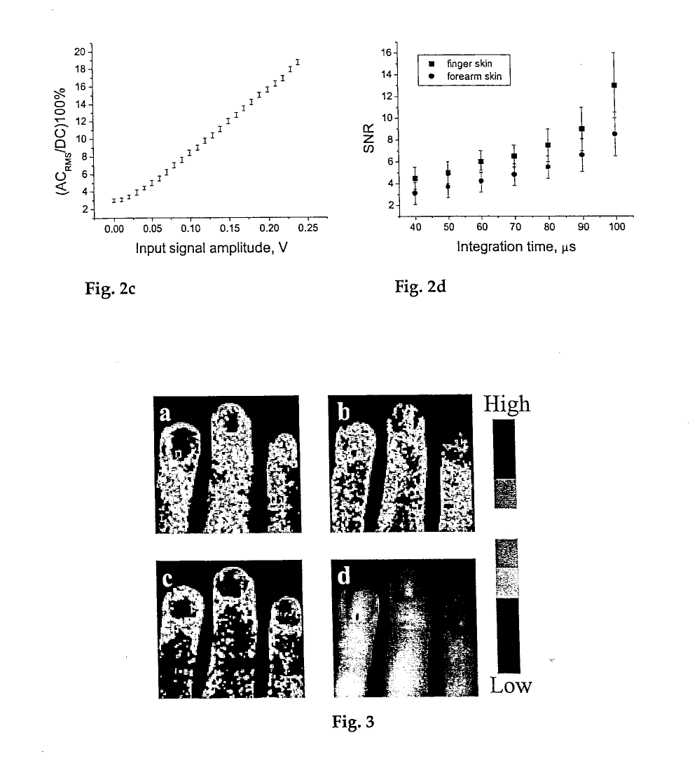

In FIG. 2(a) the moment ratio M₁/M₀ as indicated above shows the speed response of the imager as a function of the input signal frequency. The input signal of 10% modulating depth ACᵣₘₛ/DC was measured for the frequency range from 100 to 6500 Hz. A linear dependence of the moment ratio M₁/M₀ imager response is found up to the Nyquist frequency; that matches well to the theoretical expectation. Effectively, the measured (M₁/M₀)fₛ value should be equal to the signal frequency, which is clearly seen from the results. Beyond 6000 Hz, a decay in the imager response is observed due to an aliasing effect. It should be noted that the digital image sensors do not usually include antialiasing circuitry in their design; therefore the aliasing effect is virtually unavoidable in the imager. An antialiasing filter must be employed before the signal is digitized. It usually does not have an effect applying a low pass filter on the digitized signal because the aliasing effects occur before of the sampling process. Any aliasing effects would already be stored in the digitized signal and cannot be removed by low pass filtering as the effects appear as low frequencies in the signal. It should be noticed in addition, that the integrating sensor reduces the aliasing effect by suppressing the amplitude of the higher frequency components.

In FIG. 2(b) the AC_{RMS}/DC ratio of the imager as a function of the input signal frequency is shown. The AC_{RMS}/DC value is proportional to the square root of M₀ moment. The decay in the \sqrt{M₀}, imager response is due to the non-zero integration time of the detectors. This dependence is very similar to the frequency response of a basic low pass filter RC-circuit with a time constant defined by equation (9); see also equation (11). The decay of a factor of 0.5 for the RC-circuit is typical. For the integrating sensor the signal response near the cut-off frequency is even smaller and being approximately of 0.7 of its maximum.

In FIG. 2(c) the imaging system \sqrt{M₀} response to the amplitude changes of the input signal is shown. The input signal frequency was fixed at 3000 Hz. The imager signal amplitude response shows an expected linear dependence. At low amplitudes of the input signal the imager response demonstrates a nonlinearity caused by the noise.

Finally in FIG. 2(d) the SNR (signal to noise ratio) of the system for measurements on a finger and on the inner forearm skin is shown. The standard deviations for each measured values are also given.

A further object of this invention is to use different exposure times for different photo detectors or pixel areas for increasing the intra-scene dynamic range of the sensor. This is applied for measuring the samples with highly reflective parts.

A further object of this invention is to describe a Laser Perfusion Imaging system characterized by two imaging modes of operation: laser Doppler imaging (LDI) and laser Speckle Imaging (LSI). The said imaging modes are chosen depending on the requirement of a particular application. The LDI mode is characterized by higher accuracy; the LSI mode is characterized by higher speed. In the said imaging system, during the measurements the imaging mode can alter between LDI and LSI.

A further object of this invention is the use of an integrating instead of a non-integrating detector as used in the prior mentioned publications and patents.

There exist two basically different concepts in CMOS image sensor technology for capturing photons on the detector: non-integrating and integrating detectors.

In non-integrating detector, the photon flux is continuously converted into an electrical current i.e. the output signal. To obtain images, the detector array is read-out instantaneously by means of sequential photoelectrical scanning. One pixel detects only the photons that are captured during the sampling time of the pixel:

$$Δ t = \frac{{T}_{tot}}{N} . \qquad ( 1 )$$

Here Tₜₒₜ is a time to read-out all N pixels of the frame (or sub-frame). Thus, during Δt one pixel detects X photons:

$${X}_{non \_ int} ∝ \frac{{P}_{tot}}{N} Δ t . \qquad ( 2 )$$

Here Pₜₒₜ is the total illumination power.

In the integrating detector concept the total photon current is integrated as a charge when the detector captures photons. All charges are accumulated in a small capacitor, which at the end of the exposure time interval is red out. The charge is then converted into the output signal linearly proportional to the number of photons captured by the detecting pixel. In addition each pixel collects photons during the time other photo detectors are read-out (rolling shutter mode) or all photo detectors collect photons during the exposure time interval and they are read-out immediately thereafter (global shutter mode). The maximum integration time (or exposure time) is equal to the time to read-out N pixels, Tᵢₙₜ=Tₜₒₜ. Therefore, the number of photons detected by one pixel of an integrating detector array is

$${X}_{non \_ int} ∝ \frac{{P}_{tot}}{N} {T}_{tot} \qquad ( 3 )$$

For both systems the signal to noise ratio (SNR) is determined by the number of detected photons X:

SNR∝\sqrt{X} (4)

So the advantage in the SNR for the integrating system is

$$\frac{{SNR}_{int}}{{SNR}_{non \_ int}} = \sqrt{N} \qquad ( 5 )$$

Here we have compared two imaging systems, one with integrating detector array and one with a non-integrating (scanning) detector array. Up to now we have assumed equal detector noise for both imagers, which is not always true. For completing these considerations, the influence of the temporal noise on SNR of each imaging system should also be considered.

For both types of sensors, the minimum noise floor consists of thermal noise, TN, and shot noise, SN, caused by the average photocurrent plus average dark current, [symbol] I [symbol] = [symbol] Iₚₕₒₜₒ [symbol] + [symbol] I_{dark} [symbol] , in the circuit:

$$TN = 〈 {i}_{TN}^{2} 〉 = \frac{4 \cdot k \cdot T \cdot {B}_{n}}{R} \\ SN = 〈 {i}_{SN}^{2} 〉 = 2 \cdot q \cdot 〈 I 〉 \cdot {B}_{n} . \qquad ( 6 )$$

Here k is Boltzmann's constant, e is the charge of an electron, T is the temperature in degrees Kelvin, Bₙ is the noise equivalent bandwidth, and R is the load resistance. The value of the load resistance is determined by the upper cutoff frequency fₛ required to pass the signal

$$R = \frac{1}{2 π \cdot C \cdot {f}_{s}} , \qquad ( 7 )$$

where C is the capacitance of the photo detector. The signal-to-noise ratio (SNR) is then

$$SNR = \frac{〈 {i}_{s}^{2} 〉}{2 \cdot q \cdot 〈 I 〉 \cdot {B}_{n} + 8 π \cdot k \cdot T \cdot C \cdot {f}_{s} \cdot {B}_{n}} . \qquad ( 8 )$$

First, consider the non-integrating devices. In general, the noise bandwidth and the signal bandwidth are not the same. If the upper cutoff frequency is determined by a single RC time constant the signal bandwidth and the noise bandwidth are accordingly

$${f}_{s} = \frac{1}{2 π \cdot R \cdot C} \\ {B}_{n} = \frac{1}{4 \cdot R \cdot C} = \frac{π}{2} {f}_{s} . \qquad ( 9 )$$

Thus for the non-integrating detector the SNR is

$${SNR}_{non \text{-} int} = \frac{〈 {i}_{s}^{2} 〉}{π \cdot q \cdot 〈 I 〉 \cdot {f}_{s} + 4 {π}^{2} \cdot k \cdot T \cdot C \cdot {f}_{s}^{2}} . \qquad ( 10 )$$

Second, for the integrating detector, the SNR is expressed as before, equation (8), except that the noise bandwidth is now defined as Bₙ=1/(2·Tᵢₙₜ), where Tᵢₙₜ is the time interval between successive readout cycles of the diodes (the integration time or exposure time interval). The bandwidth of the Laser Perfusion Imaging system is adjusted to the measured signal bandwidth by means of setting-up the exposure time of the image sensor to a predetermined value defined by the signal bandwidth. Therefore, to match the signal bandwidth the integration time is determined by

$${B}_{n} = \frac{1}{2 \cdot {T}_{int}} = {f}_{s} . \qquad ( 11 )$$

Now we find for SNR of the integrating detector

$${SNR}_{int} = \frac{〈 {i}_{s}^{2} 〉}{2 \cdot q \cdot 〈 I 〉 \cdot {f}_{s} + 8 π \cdot k \cdot T \cdot C \cdot {f}_{s}^{2}} = \frac{π}{2} {SNR}_{non \text{-} int} . \qquad ( 12 )$$

Thus, at the same photocurrent, the SNR of the integrating detector is about a factor of 1.5 better than for the non-integrating device.

Finally, using equation (5) and equation (12) we find that for the scan case, where only one pixels of the image is measured at a time, the SNR of the integrating detector array can be increased by a factor of:

$$\frac{{SNR}_{int}}{{SNR}_{non \_ int}} = \frac{π}{2} \sqrt{N} . \qquad ( 13 )$$

The above considerations concern the fundamental difference between the detectors, however some technological features that influence the detector performance should also be mentioned.

One problem encountered in non-integrating detector is the dependence of the time constant on the signal level; that makes the non-integrating detector bandwidth to be dependent on the signal level. This problem could be in principle eliminated but on the expense of the increased noise floor caused by the on-chip integrated amplifier circuit.

As for the integrating system, an additional advantage available here is the possibility of reducing the effect of the thermal noise. This can be achieved by a well-known correlated double sampling signal processing method. Also, the read-out noise of the non-integrating sensor is usually about an order of magnitude higher than for the integrating one.

Another essential advantage of the integrating detector concept is the flexibility in selecting the integration time in order to match the required signal bandwidth. Since both shot and thermal noises are distributed over a wide frequency range, reducing effectively the noise bandwidth reduces the noise contribution of the measurement. Therefore the integration time can be used as an additional degree of freedom for an optimized high-speed Laser Perfusion Imaging system.

The Laser Perfusion Imaging system as described above, may further comprise an auto-mode operation where the optimal settings for the imaging system (gain, bandwidth, exposure time, etc) are set autonomously depending on the measured object properties (velocity, illumination conditions, etc.) and the auto-settings are determined by the object image and analysis based on flow-map images histograms but not limited to.

In FIG. 3 flow-related images obtained of a finger for a healthy person are shown. An image of 256×256 pixels was obtained with the LDI imager: FIG. 3 a) perfusion map [Low=1500 a.u.; High=3000 a.u.]; FIG. 3 b) blood concentration map [Low=150 a.u.; High=300 a.u.]; FIG. 3 c) flow speed map [Low=500 a.u.; High=1500 a.u.]; FIG. 3 d) standard digital image of the finger. The total imaging time was 3.5 seconds.

The images are obtained for the imager settings for the bandwidth from 100 to 6000 Hz with 100 Hz resolution; the integration time was 82 μs. A smoothing filter was applied to the row images: the shown value of each pixel was obtained by averaging the raw-values of 8 neighboring pixels. The flow images (perfusion, concentration, speed) are false-color coded with 9 colors. This coding is relative and does not mean that measured perfusion value coded by e.g. red is equal to the value for concentration or speed coded by the red color. The images clearly show the difference in speed and concentration distributions measured on the fingers.

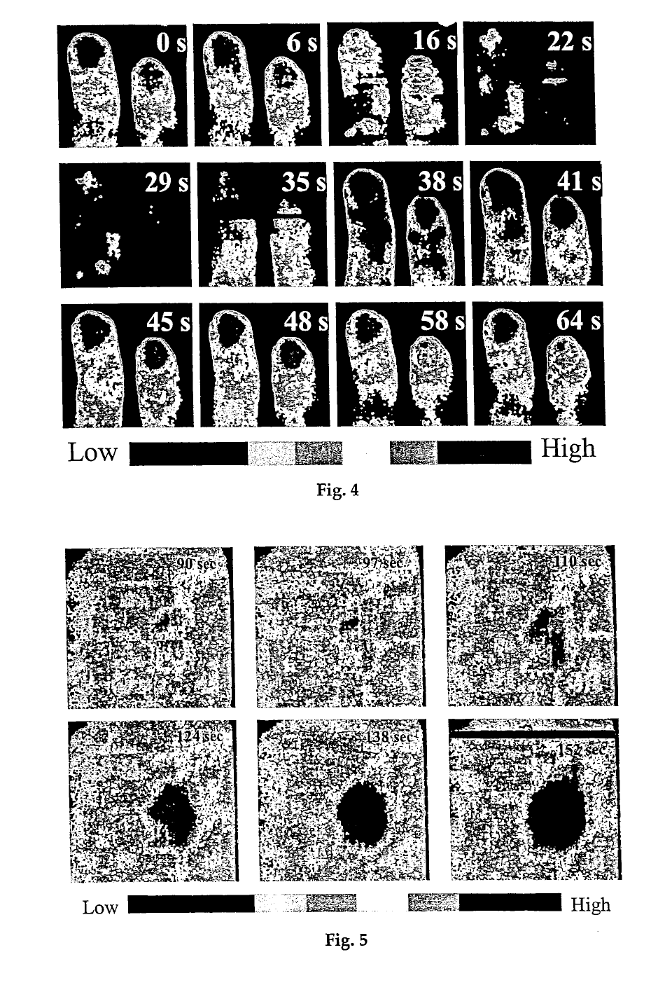

The perfusion images shown in FIG. 4 are obtained during an artery occlusion experiment. The imager settings were the same as for the measurements described in FIG. 3. This example demonstrates the performance of the imager in the continuous imaging mode. The images were taken sequentially with a time difference of 3.5 seconds, comparable to the imaging time for one image. The selected images ordered in a matrix of 4×3 images visualize the perfusion time sequence before, during and after the occlusion. As expected, there is a decrease of perfusion during the occlusion. After release of the occlusion the local perfusion shows an “overshooting” i.e. a marked increased perfusion above the initial perfusion; this physiological effect is known as reactive hyperemia. Shortly after, the perfusion returns to the initial state.

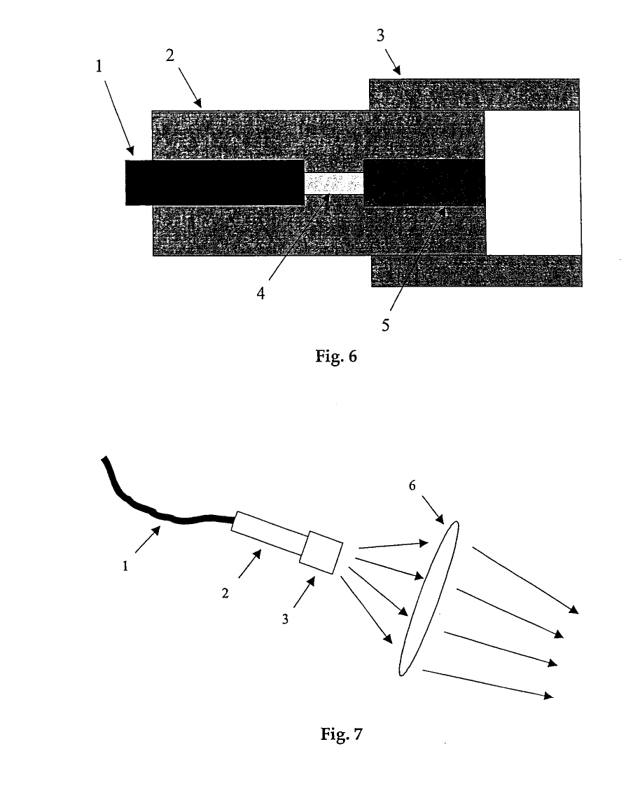

The effect of a topical applied agent is clearly seen on the images shown in FIG. 5. A small amount of this agent penetrates and crosses the skin layers and induces a perfusion increase within a few minutes. The images show the time trace of the penetration history until the heavily increased subcutaneous perfusion response.

The perfusion images (256×256 pixels) are obtained with the high-speed laser Doppler imager. The imaging area is 5.5×5.5 cm². The agent was applied on the skin of the inner side of the forearm. The perfusion images show the blood flow changes in time: in 90, 97, 110, 124, 138, and 152 seconds after the topical agent was applied to the skin. Imaging time is approx. 3.5 seconds per image. Here, the “Low” corresponds to a perfusion value of 500 [arbitrary units] and “High” to a perfusion value of 2500 [arbitrary units].

FIG. 6 shows details of the fiberized illumination device for a uniform sample illumination. This comprises an optical fiber (1), a mechanical holder (2), an outer protection ring (3), the fiber core (4), and a GRIN lens (5).

FIG. 7 shows the uniform diffuse illumination device. This comprises an optical illumination as described in FIG. 6 and a further focusing lens (4).

The present invention is not limited to visualize perfusion, flow velocities and concentration of blood particles, but is also applicable to any field where moving particles interact with coherent light and where this coherent light is superimposed with coherent light coming from non-moving particles. This is the case in water, oil, air etc where the physical i.e. convective, thermal perturbations or laminar-turbulent flow changes but not limited to these examples create particles speed or concentration distributions within the measured flow.

Laser Speckle Imaging (LSI) and Laser Speckle Contrast Analysis (LASCA) both analyze the contrast of speckle which is reduced due to movement of the blood cells. The two names shall be used as synonyms. LSI can operate on spatial and/or temporal contrast analysis. The algorithms are known to those skilled in the art.

Compared to Laser Doppler Imaging (LDI), LSI has the advantage that it allows longer integration time on the camera sensor and thus requires less brightness on the sample. This for example allows a bigger field of view with the same Laser power than LDI. On the other hand, LSI has the drawback that signal contributions for LSI are hardly discernible. Both, concentration and speed of moving particles contribute to the reduced contrast measured. Additionally, the system response is not linear to the velocity since a finite sensor integration time influences the measurement. In LSI, the non-linear response is usually modeled to calculate a flow map proportional to the perfusion.

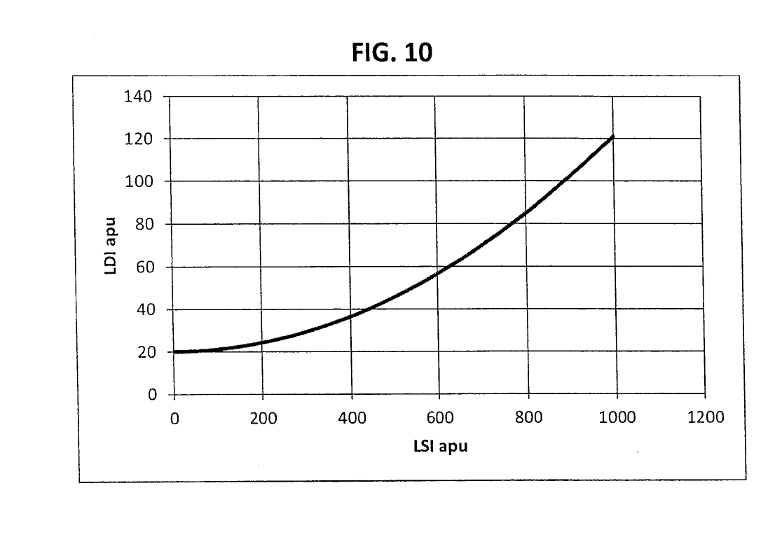

In an embodiment LDI and LSI are combined in a single device. The two modalities can operate simultaneously or sequentially and produce independent flow maps. Such combination has several advantages as the strength of the two technologies can be combined. LSI can be used to visualize a large surface while LDI is used to discriminate the signal contributions and/or to quantify them. It can usually be assumed that the speed and concentration distribution is similar in similar tissue. Thus LDI can be used to analyze the perfusion, speed and/or concentration for a sub-part of the full LSI map. LDI and LSI flow maps are taken from at least partially the same region of the object at the same or similar time intervals. The maps are then compared. The result of this matching can be used to detect more information about the signal contribution of the LSI signal. Also, LSI signal can be adjusted/calibrated based on LDI signal. Such calibration could use the LDI signal of one or several areas of the observed object and match the LSI signal of the same areas by fitting a calibration function such that the LSI signal can be converted to the LDI signal. Such calibration function could be linear or any other (normally constant) function. The fitting is normally found using a regression analysis. Thus with the fitted calibration any LSI values can be converted to LDI values (and usually inversely as well). A non-limiting example of such a function is shown in FIG. 10. In this figure the x-axis shows LSI arbitrary perfusion units (apu) and the y-axis show LDI arbitrary perfusion units (apu). The conversion function in this example is a 2^{nd} order polynomial. The structure of the calibration function is either known by design, simulated, or determined using a calibration procedure. Such procedure could be done using a phantom which can simulate different perfusion values. Because LSI has a non-linear relationship to the speed of blood the LDI information can also help to adjust the model and linearize the signal. The size and resolution of the LDI maps is usually smaller or equal to the LSI map and can also only be a single to a few pixels in width and height. Also, it is possible to use LDI on several small areas distributed over the full LSI map and/or to acquire LDI using scanning means. All combinational calculation is done in a processing unit.

In an embodiment the above mentioned calibration is further improved by using a time-series of LDI and/or LSI maps for the calibration function fitting and/or by using a phantom and fitting the function prior to the usage of the device.

In an embodiment the LSI and LDI share the same optics. In such embodiment elements of the optics can potentially be automatically or manually adjustable based on the modality (LDI/LSI) used. Such elements can be the numeric aperture of an objective or an aperture size.

In an embodiment the LSI and LDI sensors are at least two separate sensors which are illuminated through beam splitting optics or which have parallel or almost parallel independent beam paths. The integration time (i.e. the time during which the photon induced current is integrated to a charge) of those sensors is set independently. In another embodiment the same sensor is used and the integration time of such sensor is adjusted for the two modes (LDI/LSI). In yet another embodiment the integration time is kept short as needed for LDI (or even no integration exists) and the integration for LSI is done in hardware or software by summing or averaging a number of frames.

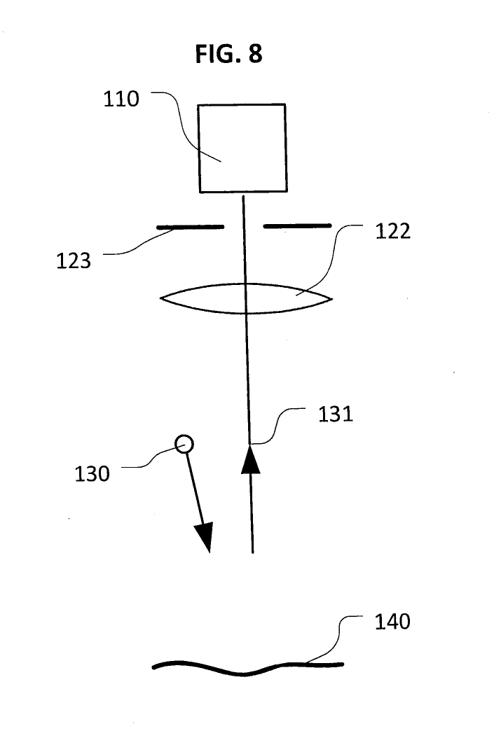

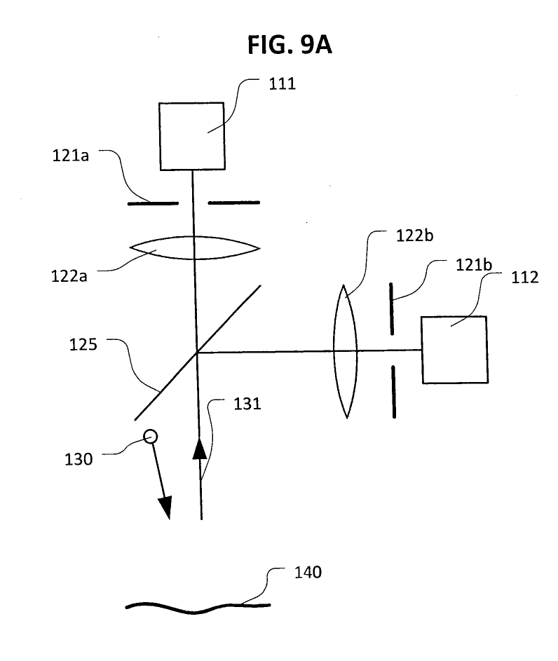

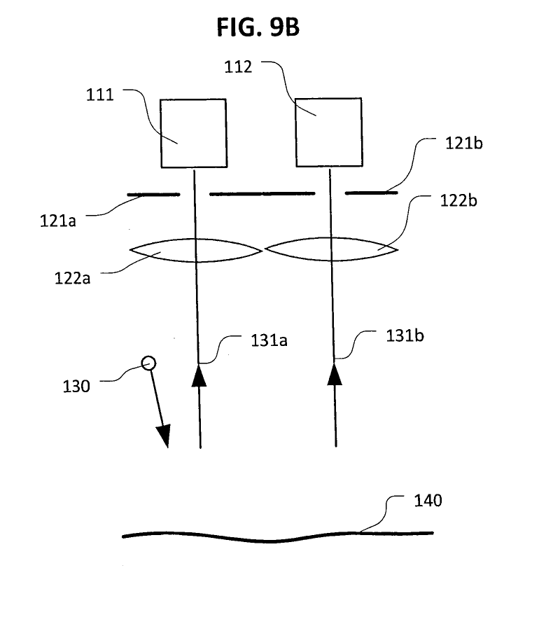

The invention will be better understood with illustrations of non-limitative examples. FIG. 8 shows a combination of LDI/LSI with a single image sensor, while FIGS. 9a and 9b show implementations with two independent sensors for LDI/LSI. The numbers on the parts have the same meaning on those three figures: The observed object (140) is illuminated with coherent light source (130). The back reflected light (131) passes through light collecting optics (122). In one embodiment the light is first split using a beam splitter (125). Usually an aperture is used before the light sensor. The aperture (121 and 123) can have a fixed size in case of separate beam path for LDI/LSI while it is preferably adjustable (123) in case of a single sensor embodiment (FIG. 8). The sequence of aperture and collecting optics can be inversed or even mixed in such way that the aperture is within the collecting optics. In case of a single sensor embodiment (FIG. 8), the sensor (110) must be suitable for both LDI and LSI signal acquisition. Such sensor has preferably a configurable integration time with a large range. In case of two-sensor solutions the sensor for LDI (111) is separated from the sensor for LSI (112). While the LDI sensor needs high frame rate, the LSI sensor needs adjustable or reasonably long integration time. The acquired signal is further passed to processing units (not shown) calculating the respective LDI and LSI signals and maps.

Another object of this invention is an auto-mode setting of the LSI integration time based on measured parameters of the LDI signal. Such auto-mode can be accomplished with analyzing one or several LDI maps (perfusion/concentration/speed) or single LDI signals. Changing the LSI integration time changes the measurable blood flow speeds. Thus the information analyzed from LDI (with methods such as histogram analysis of measured speeds or information from the power spectrum before the moment analysis) is used to determine the required speed range of LSI and thus the integration time. Model dependency between integration time, contrast and speed is non-linear and known to those skilled in the art. In the same spirit in another embodiment the LDI parameters (such as sampling frequency) is set automatically based on data from LSI (such as histogram of measured speeds).

Another object of this invention is to use LDI and LSI simultaneously or in short-interval sequence and to have LDI maps of a center region of the field of view while LSI covers a bigger field of view of the same observed object. In the center region the perfusion information is available from both modes (LDI/LSI) while in the peripheral region only LSI is available. Thus the center region can be shown with better quality and signal information (perfusion/concentration/speed/quantification) while the peripheral region only LSI based perfusion information is available. Often LSI perfusion is adjusted/scaled/calibrated to match the LDI perfusion in the center region. Such adjustment is preferably performed with linear translation such as offset and gain, but can be handled with more complex functions if needed. A new combined flow map is generated: In the peripheral region the adjusted LSI signal is shown. In the center region it is possible to show LDI signal or adjusted LSI signal only. Also, it is possible to combine the perfusion information from LDI and LSI in the center region (where data from both modes are available) by (weighted) averaging of pixels from the LDI/LSI maps from the same part of the observed object. Such method can potentially further improve the image quality.

In an embodiment the LDI and LSI maps are shown in overlaid view. Such overlay can be done with different colors/colormaps and/or semi-transparency. Also, the maps are transformed such that the pixels match the same point of the observed object. Potentially the overlay is further extended with a white light image.

Combining LSI and LDI information can help improving reliability of the data. In an embodiment the LDI and LSI maps are combined for common area by a processing unit. The processing unit adjusts the scaling of the maps or calibrates them and then compares them. Areas where the two maps match with their result (e.g. higher/lower perfusion than average or absolute value) have increased reliability while areas where the results differ have decreased reliability. The processing unit creates an addition map with the reliability information for each area. This map can have the same resolution as the maps from the Modalities or a reduced resolution. In an embodiment the above mentioned processing unit additionally produces a new perfusion map with combined information from both modalities.

In an embodiment the device operates in intra-individual relative mode, i.e. the flow maps are used relative to an acquired reference value. In such mode the flow maps no longer have absolute values, but relative values compared to a reference. Such comparison often is the ratio of the current absolute value with the reference with the relative flow map being presented in percent. But it can be any other function. In a combination of LDI and LSI the reference could be taken with both modalities independently, but from the same point of the object. Also, two independent relative flow maps are calculated with their respective reference values. The two relative flow maps can then easily be combined to a combined relative flow map using (weighted) averaging from the same part of the observed object. Also the two relative flow maps can be used to find reliability information. The approach can work with or without body mapping information (see “Body mapping of human cutaneous microcirculatory perfusion using a real-time laser Doppler imager”, Harbi 2012). With body mapping, a database of known ratio between body parts for population groups is stored and can be used to use references across body parts. The approach is proven for LDI, but most likely also works for LSI.

1. A Laser Perfusion Imaging system comprising:
at least one coherent light source configured to illuminate a selected area of interest of an object for determining flow-related data of the selected area;
a light collecting optic;
at least one image sensor including a randomly addressed 2D array of photo detectors that receive collected light intensity, a control unit;
a signal processor unit; and
a display unit configured to display results;
said system being configured to detect a laser Doppler signal and a Laser speckle signal from the selected area of interest of the object.

2. A Laser Perfusion Imaging system according to claim 1 wherein the at least one image sensor is configured to detect both a laser Doppler signal and a Laser speckle signal from the selected area of interest of the object.

3. A Laser Perfusion Imaging system according to claim 1 comprising at least two separate image sensors for laser Doppler and laser speckle signals.

4. A Laser Perfusion Imaging system according to claim 1 comprising at least one image sensor for laser speckle signal and at least one single point sensor for laser Doppler signal.

5. A Laser Perfusion Imaging system according to any of the pervious claims further comprising a processing unit which uses the detected laser Doppler signal to adjust the Laser speckle signal.

6. A Laser Perfusion Imaging system according to any of the previous claims further comprising a processing unit which calculates perfusion map and/or reliability map from a combination of LDI and LSI maps of the same observed object.

7. A method to adjust laser speckle maps based on laser Doppler signals, comprising:
acquisition of at least one laser Doppler signal and at least one laser speckle flow map simultaneously or in short sequence,
adjusting the at least one laser speckle map based on information from the at least one laser Doppler signal.

8. A method to combine laser speckle and laser Doppler maps, comprising:
acquisition of at least one laser Doppler and at least one laser speckle flow map simultaneously or in short sequence
adjusting the scale of the flow maps to align,
generating a combinational flow map and/or a reliability map by comparing the two flow maps.
